(19) United States
(12) Patent Application Publication
(10) Pub. No.: US 2008/0255659 A1
(43) Pub. Date: Oct. 16, 2008

(54) Fabrication method for drug-eluting stent with medicine-compatible loading mechanisms

(75) Inventors: Jung-Tang Huang, Taipei (TW); Pen-Shan Chao, Taipei (TW); Hou-Jun Hsu, Taipei (TW)

Correspondence Address: Jung-Tang Huang;5F., No.7, Lane 10, Sec. 2, Bade Rd., Da-an District, Taipei City 106 Taiwan (R.O.C.), Taipei, 106, TW

(73) Assignee: Jung-Tang Huang

(21) Appl. No.: 11/977,946

(22) Filed: Oct. 29, 2007

(30) Foreign Application Priority Data
Apr. 12, 2007 (TW) 096112808

(51) Int. Cl. A61F 2/04

(52) U.S. Cl. 623/1.43

(57) ABSTRACT

A MEMS-based fabrication process is disclosed to fabricate a hollow seamless drug-eluting stent. This stent fabrication process is characterized by using a photolithography process, a composite electroplating process, and a polishing process to mass-produce drug-eluting seamless stents. Combining a multi-layers photolithography process with a multi-layers composite electroforming process could make the formation of micro-holes, micro-caves, or micro-trenches integrated with this hollow seamless eluting-stent for any anti-thrombosis drug loading or filling.

### BACKGROUND

1. Field of Invention

The present invention relates to a MEMS-based (Micro-Electrical-Mechanical System) process used for making a hollow seamless stent, more specifically, relates to a process involving in utilizing a photolithography process, an electroforming process, a polishing process, an electrolytic polishing process, a material shaping process, and a drug immersing or coating process to form a drug-eluting stent. This new drug-eluting process characterizes low-cost, fine-pitch, and mass-production; and in particular, the drug-eluting stent itself could load various micro-trenches, micro-caves, or even micro-holes varying in sizes and types for anti-thrombosis drug eluting.

2. Description of Related Arts

Cardiovascular disease, mostly caused by some disorders such as high blood pressure, diabetes, smoking, high blood fat, and people with a family history of heart disease, is the most common health problem worldwide. In general, the related cardiovascular diseases in the medical clinical trial could be divided into four different types including stable angina pectoris, unstable angina pectoris, acute myocardial infarction, and sudden death. The acute myocardial infarction accounts for the highest death rate by up to 20%.

An estimated of one-third of cardiovascular patients must undergo the surgery of stent transplant. Stent, referring to a blood vessel supporter frequently made of metals, stainless steels or other kinds of specific biomedical materials, is extensively used to expand the clogged blood vessel for giving a smoother blood stream.

Laser cutting machining among other processes feasible for stent fabrication is viewed as the most commonly used process worldwide. Both etching and sputtering are the other two alternative choices for stent fabrication. Additionally, stent cut by CNC (Computer Numerical Control) machining has also been disclosed in a Taiwanese patent, where such thin metals or alloys proven harmless to human body as stainless steels, Ti (Titanium), Mg (Magnesium), or Mg-related alloys have been used to be raw materials for stent. In this process, either rolling or bending process must be followed by the spot welding process, aimed for making the both ends of the stent bonded together. The last step is to use an electrolytic polishing process to get rid of burrs arose by mechanical machining from the whole stent.

The following two examples illustrate the previous works for stent formation. In U.S. Pat. No. 2004/0162605 A1, entitled “Stent Fabrication Method”, describes a process based on the combination of a laser cutting machining process and an etching process. The stent cut from the thin metal plate either by laser cutting machining or by etching is then deformed by bending in order to make the both ends of the stent touched together. Afterwards, the deformed edges are connected at least at one point by using a spot welding process. Finally, employ a mechanical or a chemical polishing process to polish the surface of the stent for the purpose of getting a burr-free surface. Partly because it needs to request to a higher budget to purchase laser cutting machining equipment and partly because the grid density of the stent is naturally limited to the laser spot size, this process is almost impossible to cut the cost in manufacturing while enhance design capability for stent in shapes or profiles.

In U.S. Pat. No. 2003/0159920 A1, entitled “Thin Film Stent”, discloses a process involving some sacrifice layers (release layers) like oxide or nitride and employing a popular lift-off technique or an etching procedure, both are widely used in MEMS, to form the main skeleton of the stent. The ion sputtering process is subsequently used to deposit the fundamental structure of the stent. The ion sputtering process, in essence, is not ideal for stent fabrication due to far lower deposition rate. That is, this proposed method has difficult dealing with a stent in higher thickness or a substrate in larger size.

Drug-eluting stent, a term given to describe a stent partly or wholly covered with some bio-compatible anti-thrombosis drug, has been proven to have a noteworthy effect against clogged or narrowed blood vessels. Therefore, we could realize that the high incidence of clogged or narrowed blood vessels in cardiovascular disease contributes to a high demand for drug-eluting stents. The following examples illustrate some patents regarding the applications of drug-loading mechanisms in some drug-eluting stents.

In U.S. Pat. No. 7,135,038 B1 entitled “Drug Eluting Stent” indicates a drug-loading process used to fabricate a drug-loading mechanism about how to load drugs on the stent. It could form either single or multiple trenches on the surface of the stent for drug filling. Both thickness and cross-section area of the trench is slightly thinner and slimmer than those of the stent. The trench is presumably formed with a chemical etching process. A kind of polymer is then used as a medium binder to connect the drug with the surface of the stent by using a spin coating process ahead a heating process. The coated drug is said to be a great effect on curbing the recurrence of clogged or narrowed blood vessels. However, this process adopting an additional process or we call it a secondary manufacturing step to fabricate the drug-loading mechanism, trenches, rather than fabricate them simultaneously with the main structure of the stent may lead to a higher cost and more complicated manufacturing procedure.

In U.S. Pat. No. 2004/0133270 A1 entitled “Drug Eluting Stent and Methods of Manufacture” gives a drug-loading mechanism on a Ni—Ti stent or a stainless stent made from some shape memory alloys. The stent is mainly cut by using drawing, which is a process easily to form some hollow holes in the midst of the stent. Drug is then coated onto those hollow holes passing through the stent with some specific micro-syringes, and then seals the two ends of the hollow holes already filled with drug by using a spot welding process. Later, employ the laser cutting machining to carve out several tiny holes to act as the so called drug-releasing exits to release the coated drug. The stent after finishing the formation of the drug-loading mechanism is finally formed to be a three-dimensional hollow structure by the following two processes, bending and spot welding. How to accurately position the micro-syringes to the drug-loading areas to fill them up with drug is not an easy task. What's more, the two ends of the stent seamed by using a spot welding process is liable to cause an excessive concentration of stress on the welding joints, which might badly damage the connected blood vessel and reduce the bio-compatible ability between the implanted stent and the wall of the stent.

In U.S. Pat. No. 2005/0074545 A1 entitled “Stent with Improved Drug Loading Capacity” discloses another process in term of drug-eluting stent. This method is basically slightly modified from the laser cutting machining, where it mainly inserts two special types of molds into the axel of the stent to cast plenty of micro-holes for drug coating. A polymer is subsequently coated on the medium between the stent and the applied drug to reach a drug delayed-release mechanism. The method is similar to U.S. Pat. No. 7,135,038 B1, both of which increase their process complexity since they require using an extra process to form the drug-loading areas.

In conclusion, adopting an additional process or a secondary process to form the drug-loading mechanisms like micro-holes, micro-caves or micro-trenches would not only increase process complexity but also add manufacturing cost. By contrast, this present invention allowed fabricating all kinds of drug-loading mechanisms like micro-holes, micro-caves, or micro-trenches for anti-thrombosis drug together with the base of the stent, is a simple, low-cost, fine-pith, and high-versatility process for a drug-eluting stent.

### SUMMARY

The present invention is to provide a MEMS technology to mass produce a seamless drug-eluting stent capable of loading such numerous drug-coated mechanisms as micro-holes, micro-caves, or micro-trenches. This novel manufacturing method easily combines nano technologies to elute any bio-compatible anti-thrombosis drugs, making drug-eluting stent low cost, high versatility, and fine pitch. By using three major processes including a multi-layers photolithography process, a multi-layers electroforming process, and a subtle polishing process gives to fabricate sophisticated drug-eluting stents in mass production.

The advantages of low cost, fine pitch, and varied shapes benefit from the multi-layers photolithography process, by which it gives the benefit of fabricating future much thinner and finer stents as combined with a micro-electroforming process.

Unlike some given traditional manufacturing processes to stent, this invention is unnecessary to use the spot welding process to bond the both ends of the stent together. Furthermore, costing less money in manufacturing and designing, the present invention could dramatically avoid the occurrence of any defects or material deformation resulted from high temperature in the spot welding process.

Lacking for any defects and deformation is good for fabricating some specific stent having smaller dimension in its grid gap. By comparison, some traditional stent manufacturing methods like etching or sputtering generally rely on the spot welding process to bond the both ends of the stent together, making much smaller and finer stents impossible.

In addition to giving a lower manufacturing cost contributed by a mass production, this process is easy to carry any drug-loading mechanisms such as micro-holes, micro-caves, or micro-trenches for bio-compatible anti-thrombosis to prevent the recurrence of clogged and narrowed blood vessels by releasing the coated drug on the stent.

The invention provides a MEMS-based process for making a seamless integrated drug-eluting stent with capable of carrying a wide range of drug-loading mechanisms by using the steps of: (1) depositing two thin metallic layers such as Cr (Chromium) and Cu (Copper) on the surface of the substrate to serve as UBMs (Under Bump Metallurgy); (2) coating the first layer and the second layer of PRs (Photoresist) to the desired thickness and then conducting a series of photolithography processes like exposing, baking, and developing to define the patterns of the upper layer, main base, and the bottom layer of the stent for electroforming; (3) filling the opening areas left on the substrate after developing by the electroforming process; (4) utilizing a simple polisher similar to CMP (Chemical Mechanical Polishing) in terms of polishing mechanism to level off the non-uniform coplanarity and rugged electroformed surfaces; (5) turning a flat rectangular hollow stent into a three-dimensional circle hollow one prior to carrying out a heat treatment and getting rid of the burrs on the stent by using the electrolytic polishing process; (6) coating either a single-layer or multi-layers bio-compatible materials made from Au (Gold), Re (Rhodium), DLC (Diamond-like Carbon), or their combination mixed with polymer on the stent both inside and outside by immersing, composite electroforming or CVD (Chemical Vapor Deposition); (7) removing the remaining PR layers and UBMs off possibly with the help of a supersonic by putting the substrate into a given stripper or other chemical like acetone.

### BRIEF DESCRIPTION OF DRAWINGS

The detailed drawings of this invention will be fully understood from the following descriptions wherein:

FIGS. 1A-1M are schematic diagrams showing the drug-eluting fabrication process step by step.

FIGS. 2A-2D are examples showing four different mask designs.

FIGS. 3A-3D demonstrate four different types of drug-loading mechanisms on the stents.

FIG. 4 shows a schematic diagram of a seamless drug-eluting stent after a material shaping procedure.

### DETAILED DESCRIPTION

Referring to the attached drawings, a preferred embodiment of the fabrication method for drug-eluting stent with medicine-compatible loading mechanism will be fully understood in detail as follows:

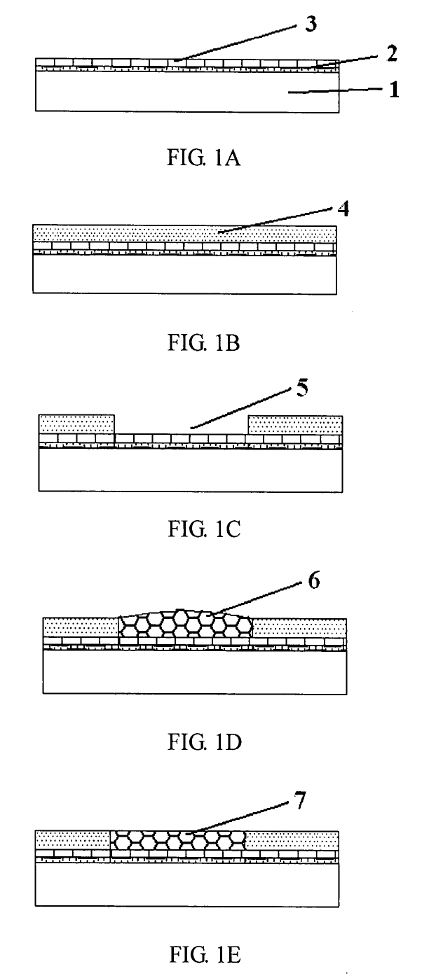

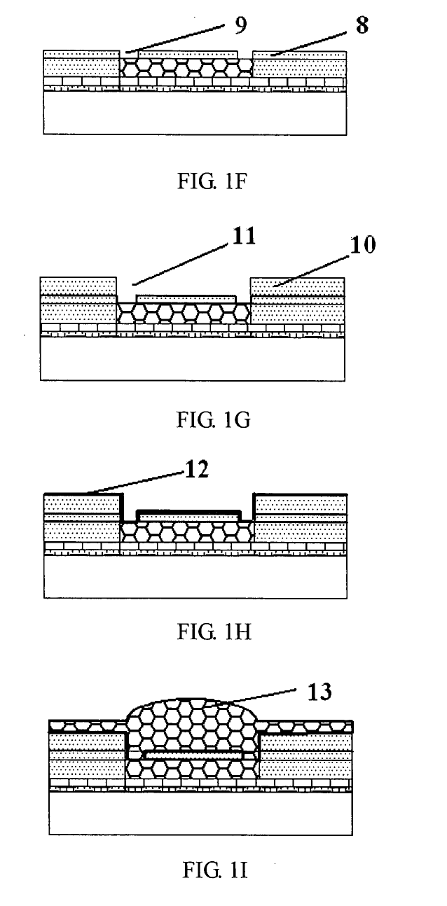

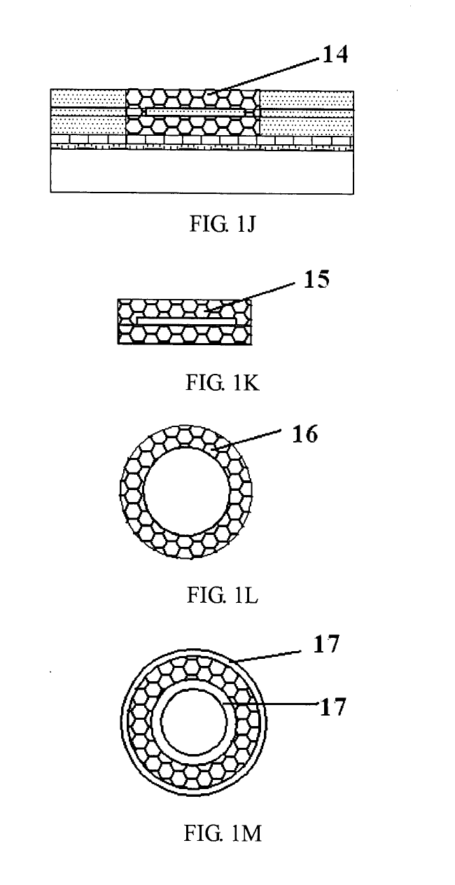

Referring to FIGS. 1A-1M, these drawings particularly present the schematic sectional diagrams step by step for the drug-eluting stent process.

Referring to FIG. 1A, firstly, two metallic layers, Cr 2 and Cu 3, are evenly coated over the substrate 1 as two UBMs by sputtering, evaporation or other deposition techniques. Other metals such as Ti (Titanium), W (Tungsten), Ni (Nickel) and Au are also available to be used as these two UBM layers.

Referring to FIG. 1B, next, the first PR layer 4 is then coated on the second Cu 3 UBM layer. The first PR layer 4 coating in the photolithography process contains three fundamental operation steps described as follows: (1) prop adequate amount of PR layer 4 on the center of the substrate 1 until the whole substrate 1 is covered at least ⅔ areas; (2) Select a lower coating speed to spread the PR layer 4 uniformly over the substrate 1 in order to acquire a desired PR layer 4 thickness; (3) Select a higher coating speed to achieve a more uniform and smooth PR layer 4.

The composition of the first PR layer 4 may have different choices from a positive tone, a negative tone, to a polyimide, and the only concerned issue is their viscosity, largely determining thickness and sidewall's angle of the PR 4 layer.

Referring to FIG. 1C, an ultraviolet light ranging from 200-450 nm is employed to directly expose the patterns made of first PR layer 4 on the substrate 1. Afterwards, the exposed substrate 1 is immersed into the PD 523 developer for few minutes to reveal the first layer of exposing area 5 (the other numerous exposing areas are not shown in this figure) designated for electroforming, and the developer should be regularly stirred to assure of getting a fully-developed substrate 1. The substrate 1 is highly recommended to be continually washed with DI water (Deionized Water) for one minute to avoid an unnecessary chemical reaction resulting from the usage of the developer and the later electroforming solution.

Referring to FIG. 1D, the first layer of exposing area 5 is then deposited to a thickness out of the first PR layer 4 or at least has the same level with the first PR layer 4. Electroform some metals, alloys, or composite materials to form the downside structure 6 of the drug-eluting stent. The possible choices for the materials are pure Ni (Nickel), Ni—Co (Nickel-Cobalt), Ni—Fe (Nickel-Iron), Fe—Co—Ni (Iron-Cobalt-Nickel) and Cr—Fe—Ni (Chromium-Iron-Nickel). Prior to performing the electroforming process, removing the oil, grease, oxide, and other contaminant with 3-5% dilute H₂SO₄ for few minutes from the substrate 1 could ensure getting a better bonding adhesion between the substrate 1 and the downside structure 6 of the drug-eluting stent. Post-treatment after electroforming is to immerse the substrate 1 into the sodiumphosphate solution for at least 30-60 s at a temperature ranging from 80° C. to 90° C. to neutralize the chemical residue left on the deposited surface 6 and dry it with N₂.

Referring to Table 1, an electroforming chemical composition for the Cr—Fe—Ni trio metal is listed.

TABLE 1

| Operation conditions | Contents (g/l) |
|---|---|
| Chromium chloride | 250 |
| Ferric chloride solution | 46 |
| Ferrous chloride | 30 |
| Sodium citrate | 70 |
| Aluminum chloride | 130 |
| PH | 0.2-0.3 |
| Temperature (° C.) | 30 |
| Electric current density (A/dm²) | 25-30 |
| Anode electric current efficiency (%) | 10-24 |
| Contents of anode metals' Fe/Ni/Cr (%, wt) | 3-29 Cr |
| | 8-54 Ni |
| | the rest content is Fe |

Referring to FIG. 1E, the downside structure 6 of the drug-eluting stent is subsequently planarized with a simple polisher to change it into a flat and uniform downside structure 7. The sequences of this polishing process are described as following: (1) Polishing pad pre-wet, a process to rinse the soft polishing pad before the start of each polishing action, is employed under the flow rate at 300 ml/min until the soft polishing pad is totally cleaned; (2) Then, the substrate 1 is mounted on the specific polishing fixture; (3) Set the polishing velocity and polishing time to the desired ranges; (4) The flow rate of the polishing slurry ejected from the spray nozzle is set preferentially between 150 to 200 ml/min; (5) Employ a nylon brush along with the spread of DI water to scrub the surface of the soft polishing pad to prevent the polishing slurry from sinking into the soft polishing pad; (6) Finally, the substrate 1 is rinsed with little DI water and dried with N₂ to avoid being contaminated by unclear particle out of the air or the holding gadgets.

Referring to FIG. 1F, which, in essence, repeats the previous processes from FIG. 1B to FIG. 1E illustrates the fabrication of the middle net structures used to connect the downside structure 7 and the topside structure 14 of the drug-eluting stent. The S1813, a positive tone PR with lower viscosity, could be used to create a second PR layer 8 as thin as up to 2 um. After developing, the opining areas 9 reserved for electroforming would appear.

Referring to FIG. 1G, the third PR layer 10 is then coated on the second PR layer 8 to define the topside structure 13 of the drug-eluting stent. This third PR layer 10, in general, has the same thickness as the first PR layer 4, close to 80 um. Similarly, the opening areas 11 for the metal deposition are developed after a series of photolithography processes. Actually, the opining areas 11 would locate at the same position as the opening areas 9 on the second PR layer 8.

Referring to FIG. 1H, a third UBM layer 12 is coated on the third PR layer 10 to form the joint metal layer between the downside structure 7 and the topside structure 14.

Referring to FIGS. 1I-1J, the third UBM layer 12 is served as a conductive metal layer for the purpose of depositing the topside structure 13 of the drug-eluting stent. This topside structure 13, treated with the same procedures as used for the downside structure 7, is later changed into a uniform and flat topside structure 14 after finishing the polishing process.

Referring to FIG. 1K, all of the three PR layers 4, 8, and 10 together with the Cr 2, and Cu 3 two UBMs are stripped from the substrate 1 by using acetone and some given PR stripper in a supersonic tank to turn into a flat rectangular hollow stent 15.

Referring to FIG. 1L, the flat rectangular hollow stent 15 is then carried out a heat treatment at a temperature 110° C. This procedure called annealing would give the flat rectangular stent 15 to have a material structure similar to 316 L stainless steel. After the electrolytic polishing and a material shaping process, generally formed by bending or rolling, a circle hollow stent 16 would be formed in place of the flat rectangular hollow stent 15.

Referring to FIG. 1M, the circle hollow stent 16 is later coated a good bio-compatible layer 17 both inside and outside by immersing in the electroforming tank filled with Au or Re chemical solution. An alternative method is to use the CVD process to cover the whole circle hollow stent 16 with a DLC or a TiO₂ (Titanium Oxide) layer. Besides, combining a layer mixed with polymer and anti-thrombosis drug outside the diamond-like or the TiO₂ layer to form the bio-compatible layer 17 is also workable.

The electroforming equipment is made up of a power supply, a chemical solution tank, a heater, an DI water supply system, and a temperature feedback system.

Using the composite electroforming process could introduce a second phase material substance absolutely different from the main electroformed structures to improve the related material characteristics, hardness or toughness is particularly. The second phase materials added into the main electroformed structures may contain some ceramic powder like Al₂O₃ (Aluminum Oxide) or SiC (Silicon Carbide), graphite, Teflon, diamond, and diamond-like carbon.

The deposition mechanism of the composition electroforming basically contains two different phrases. The first phase is called the physical absorption process, where the added particle is loosely attracted on the cathode. By contrast, the second phase that could produce a stronger absorption between the particle and the anode by an electric field effect is called the chemical absorption process. Using the composite electroforming contains the advantages of (1) improving the hardness of the deposited structure; (2) giving a better wetting angle (3) increasing corrosion resistance ability; (4) achieving the surface treatment; and (5) enhancing the material characteristics of the deposited structure. The composite electroforming process could share the same electroforming equipment as the regular electroforming process to deposit the second phase materials into the main structures of the drug-eluting stent. Some bio-compatible anti-thrombosis drugs are also possible to be externally coated on the combination of the composite materials and the main deposited electroforming structures.

Another alternative method of coating the bio-compatible anti-thrombosis drugs is to coat them on the stent base directly after the electroforming process to reach the drug-coated effect. Besides, previously coating a medical-friendly powder onto the anode before the electroforming process to make it dissolved with the electroforming chemical solution in which some additives or supplements are added is also feasible.

ZnO (Zinc Oxide), a piezoelectric material with a better bio-compatible characteristic, could be mixed with some nano-scale powder like Al₂O₃, SiC, TiO₂, or WC (Tungsten Carbide) for the composite electroforming process to form a protective layer both inside and outside of the stent.

In the polishing process, the polishing pad could be divided into two different types, a hard plate and a soft plate. The former is made up of stainless steel, while the later is composed of non-woven or polyurethane. The polishing slurry used to polish the electroformed material includes Al₂O₃ or SiO₂.

The electrolytic polishing process is beneficial to modify the right angle around the corner of the stent into an arc angle and improve the overall surface roughness. The surface roughness of the stent would be fined after the electrolytic polishing process, and the improved surface roughness could provide a better interface between stent and polymer for drug coating.

Kept in a low concentration, some metals such as Fe, Cr, Co, Ni, Ti, Ta (Tantalum), Mo (Molybdenum), and W could be safely used either in the artificial organs or in the implanted devices. Generally, Fe and Co are two essential elements to form red blood cells and vitamin B complex, B₁₂ is particularly. However, in some cases, the erosion caused by blood may get the artificial organs or implanted devices peeling off with the increase of time. The bio-compatible characteristic of these medical devices would be debilitated, which would pose a great threat to human body. That means how to increase the anti-rust ability for the implanted devices is a prime concern regarding the safety for the patients.

It has been proven that the 316 L stainless steel has the best bio-medical characteristic, largely with coming from its two major compositions, Cr and Ni. Cr could form a stable chromic oxide when reacted with oxygen, even though it is an active element. The chromic oxide is a good rust-proof substance. Likewise, Ni is also famous for producing a better characteristic against rust. Some other stainless steels containing Mo could particularly prevent from pitting corrosion in the salt solution; further, the anti-rust characteristic against chloride could be notably advanced by lowering the carbon level in those stainless steels.

The surface of the stainless steel treated by the electrolytic polishing process could obtain better material characteristics including leveling, passivation, and lightness, all of which could bring some advantages in terms of chemical passivation, chemical stable, anti-rust characteristic, free-poison interface, and free-from carcinogen.

After some regular metal machining or metal carving, workpieces are liable to produce burrs on the corner areas or near the sidewall of the structures. The mechanical polishing is suitable to get rid of those burrs especially for those with larger dimension size, but it is not workable for some tiny structures like stent. Therefore, the electrolytic polishing process is the best choice for stent polishing.

Such the devices implanted into the human body for a long time as stents, temporary anchorage devices, and artificial joints generally have a close relationship with blood, bones, and organisms. They naturally have very strict requirements for bio-compatible characteristic and cleanliness. The rougher surface roughness tends to produce more burrs, which often cause some deadly thrombus to blood vessels; worst of all, the non-uniform surface could also affect the functionality of the other organs in the human body. The electrolytic polishing process enables to get a finer surface roughness with no limitation to the shapes of the workpiece.

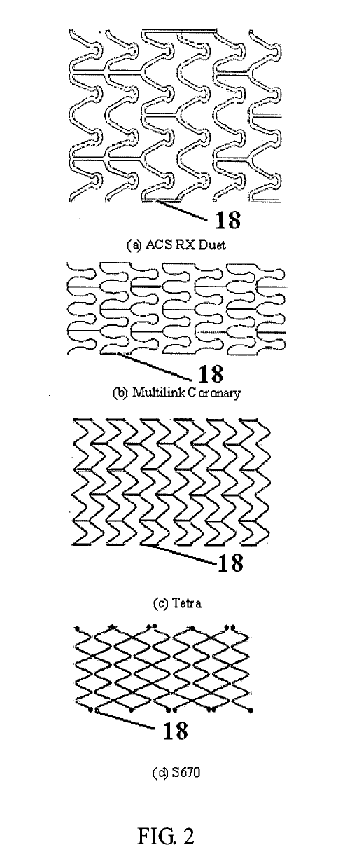

Referring to FIGS. 2A-2D, these four pictures show the patterns of the stents from the models of ACS RX Dute, Multilink Coronary, Tetra, and S 670. This invention is particularly suitable to make symmetric stents once the symmetric line 18 is specifically defined on the mask. With giving the layout of this symmetric line 18 on the mask, stents like those above-mentioned stent models could be easily fabricated.

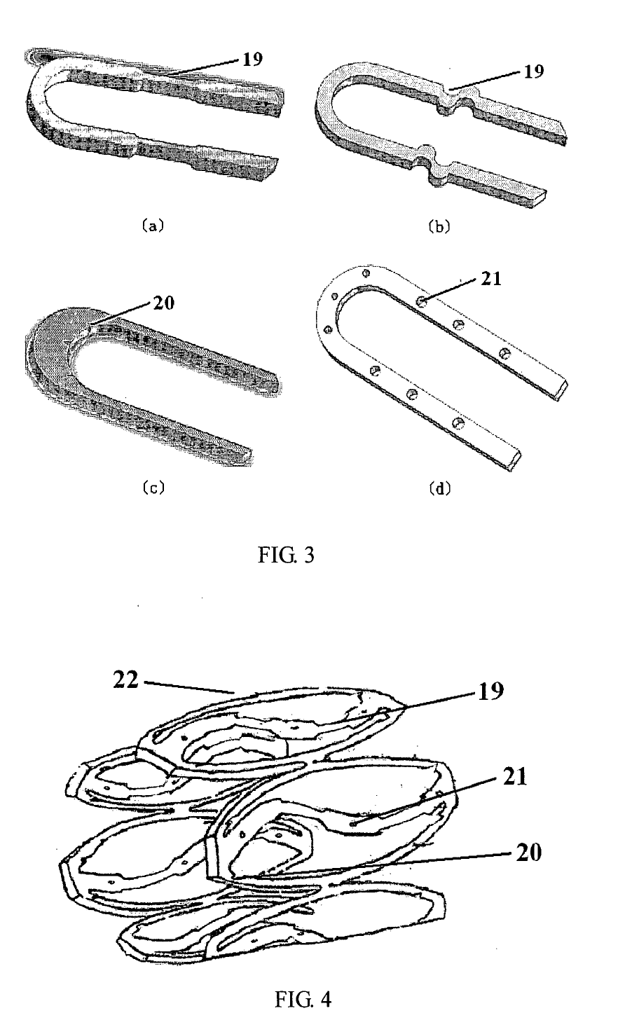

Referring to FIGS. 3A-3D, they primarily depict the drug-loading mechanisms on the electroformed surface. This invention, according to different design in masks, is readily to form the variety of drug-loading mechanisms such as micro-trenches 19, micro-caves 20, and micro-holes 21.

Referring to FIG. 4, it is a schematic diagram of a profile-shaped drug-eluting stent 22 with carrying the drug-loading mechanisms of micro-trenches 19, micro-caves 20, and micro-holes 21.

Although the invention has been described with reference to particular embodiments, the description is only an example of the invention's application and should not be taken as a limitation. Various adaptations and combinations of features of the embodiments disclosed are within the scope of the invention as defined by the following claims.

What is claimed is:

1. A method for making drug-loading mechanisms on a drug-eluting stent to carry bio-compatible anti-thrombosis drug is based on a photolithography process, an electroforming process, a polishing process, a material shaping process, and an electrolytic polishing, comprising the steps of:
(1) defining the layout of the stent by masks or stencil printings;
(2) transferring the layout of the stent to a substrate by using a photolithography process;
(3) employing an electroforming process to deposit metals, alloys, or composite materials on the stent;
(4) using a polishing process to polish the electroformed surface of the stent;
(5) taking the polished stent off the substrate and shaping it into a circle hollow stent by some cold working processes like bending or rolling; and
(6) utilizing an electrolytic polishing process to clean burrs away from stents.

2. The method of claim 1, wherein a coating process or an immersing process is followed by the electrolytic polishing process to deposit a layer with capable of loading bio-compatible anti-thrombosis drug with the stent both inward and outward.

3. The method of claim 1 wherein in said step (1), the designed stent comprises such many varied structures aimed for loading any bio-compatible anti-thrombosis drug as micro-holes, micro-caves, or micro-trenches.

4. The method of claim 1 wherein in said step (2), some metals or non-conductive materials such as silicon wafers, glass, and rubber are choices of substrates for stent pattern transfer.

5. The method of claim 1 wherein in said step (3), the electroforming process in accordance with the different layout and design of the stent is repeatable. Using a multi-electroforming process enables to deposit micro-holes, micro-caves, or micro-trenches drug-loading mechanisms with different thicknesses, giving the stent more flexible and diversified in non-uniform thickness design.

6. The method of claim 1 wherein in said step (3), the alloys for making stents could be pure Ni, Ni—Co, Ni—Fe, Fe—Co—Ni, or Cr—Fe—Ni.

7. The method of claim 1 wherein in said step (3), the composite materials for making stents could be ceramic powder like Al₂O₃ or SiC, or other materials like graphite, Teflon, or diamond-like carbon.

8. The method of claim 1 wherein in said step (4), the polishing process could improve the surface roughness of the stent while control its thickness after electroforming.

9. The method of claim 8 the polishing process is performed by using a polisher, which is made up of a polishing platen, a hard polishing plate, a soft non-woven polishing pad taped on the had polishing plate, a loading pressure, and a substrate holding fixture.

10. The method of claim 1 wherein in said step (5), such various cold-working processes as bending, rolling, lapping, or expanding could assist in shaping the stent into the desired shape compatible with Percutaneous Transluminal Coronary Angioplasty (PTCA).
